(19) United States
(12) Patent Application Publication
(10) Pub. No.: US 2018/0264125 A1
(43) Pub. Date: Sep. 20, 2018

(54) GLYCOPEPTIDE COMPOSITION

(71) Applicant: SeeCure Taiwan Co., Ltd., Kaohsiung City (TW)

(72) Inventors: Ning Tsao, Kaohsiung City (TW); Wei-Chung Chang, Kaohsiung City (TW)

(73) Assignee: SeeCure Taiwan Co., Ltd., Kaohsiung City (TW)

(21) Appl. No.: 15/894,897

(22) Filed: Feb. 12, 2018

Related U.S. Application Data
(60) Provisional application No. 62/471,985, filed on Mar. 16, 2017.

(51) Int. Cl. A61K 47/64

(52) U.S. Cl. CPC A61K 47/64

(57) ABSTRACT

A glycopeptide composition including a plurality of glycopeptides and a therapeutic agent or diagnostic compound is provided. Each of the glycopeptide includes a polysaccharide moiety connected to a peptide moiety, and the polysaccharide moiety is covalently bounded to the peptide moiety via a fixed connection point, wherein the fixed connection point is the same in each of the glycopeptide, and the therapeutic agent or diagnostic is conjugated to each of the glycopeptide.

### CROSS-REFERENCE TO RELATED APPLICATION

This application claims the priority benefit of U.S. provisional application Ser. No. 62/471,985, filed on Mar. 16, 2017. The entirety of the above-mentioned patent application is hereby incorporated by reference herein and made a part of this specification.

### BACKGROUND

### Technical Field

The present invention generally relates to a glycopeptide composition, in particular, to a glycopeptide composition containing polysaccharides connected to peptides.

### Description of Related Art

Glycopeptides may generally be used as drug delivery vehicles or even as drugs themselves. However, the problem with conventional glycopeptides is that the number of saccharide units cannot be appropriately controlled, and its connection point to the peptides are unknown. For example, when coupling peptides (poly(glutamic acid)) to polysaccharides (poly(glucosamine)), due to the vast amount of free acid on poly(glutamic acid) and the vast amount of free amine on poly(glucosamine), the glycopeptide synthesized may be connected in a wide variety of different ways, which is undesired for drug design. In order to obtain glycopeptides having the desired characteristics for drug delivery, or be used as biomaterials, there is a need to improve the current techniques for synthesizing glycopeptide compositions.

### SUMMARY

Accordingly, the present invention is directed to a glycopeptide composition where the number of saccharide units can be appropriately controlled, and the plurality of glycopeptide synthesized are all connected in the same way.

In accordance with one embodiment of the invention, a glycopeptide composition including a plurality of glycopeptides and a therapeutic agent or diagnostic compound is provided. Each of the glycopeptide includes a polysaccharide moiety connected to a peptide moiety, and the polysaccharide moiety is covalently bounded to the peptide moiety via a fixed connection point, wherein the fixed connection point is the same in each of the glycopeptide, and the therapeutic agent or diagnostic is conjugated to each of the glycopeptide.

In an embodiment of the invention, the peptide moiety is poly(glutamic acid) or poly(aspartic acid).

In an embodiment of the invention, the polysaccharide moiety is poly(glucosamine) and the peptide moiety is poly(glutamic acid).

In an embodiment of the invention, the poly(glucosamine) is connected to the poly(glutamic acid) via an amide bond.

In an embodiment of the invention, a number of glucosamine units in the poly(glucosamine) is in a range from 2 to 8.

In an embodiment of the invention, a number of glutamic acid units in the poly(glutamic acid) is in a range from 3 to 20.

In an embodiment of the invention, the poly(glucosamine) has four glucosamine units and the poly(glutamic acid) has ten glutamic acid units.

In an embodiment of the invention, when the poly(glucosamine) has n glucosamine units, then an amount of protected amine groups on the poly(glucosamine) is n−1.

In an embodiment of the invention, only one free amine group on the poly(glucosamine) is available for coupling to the poly(glutamic acid), and only one free acid group on the poly(glutamic acid) is available for coupling to the poly(glucosamine).

In an embodiment of the invention, the one free acid group on the poly(glutamic acid) is located on a C-terminal of the poly(glutamic acid), such that the poly(glucosamine) is covalently bounded to the C-terminal of the poly(glutamic acid) via an amide bond.

In an embodiment of the invention, after deprotecting the glycopeptide, the glycopeptide is one compound selected from the list of compounds represented by formula (1A) to formula (1D):

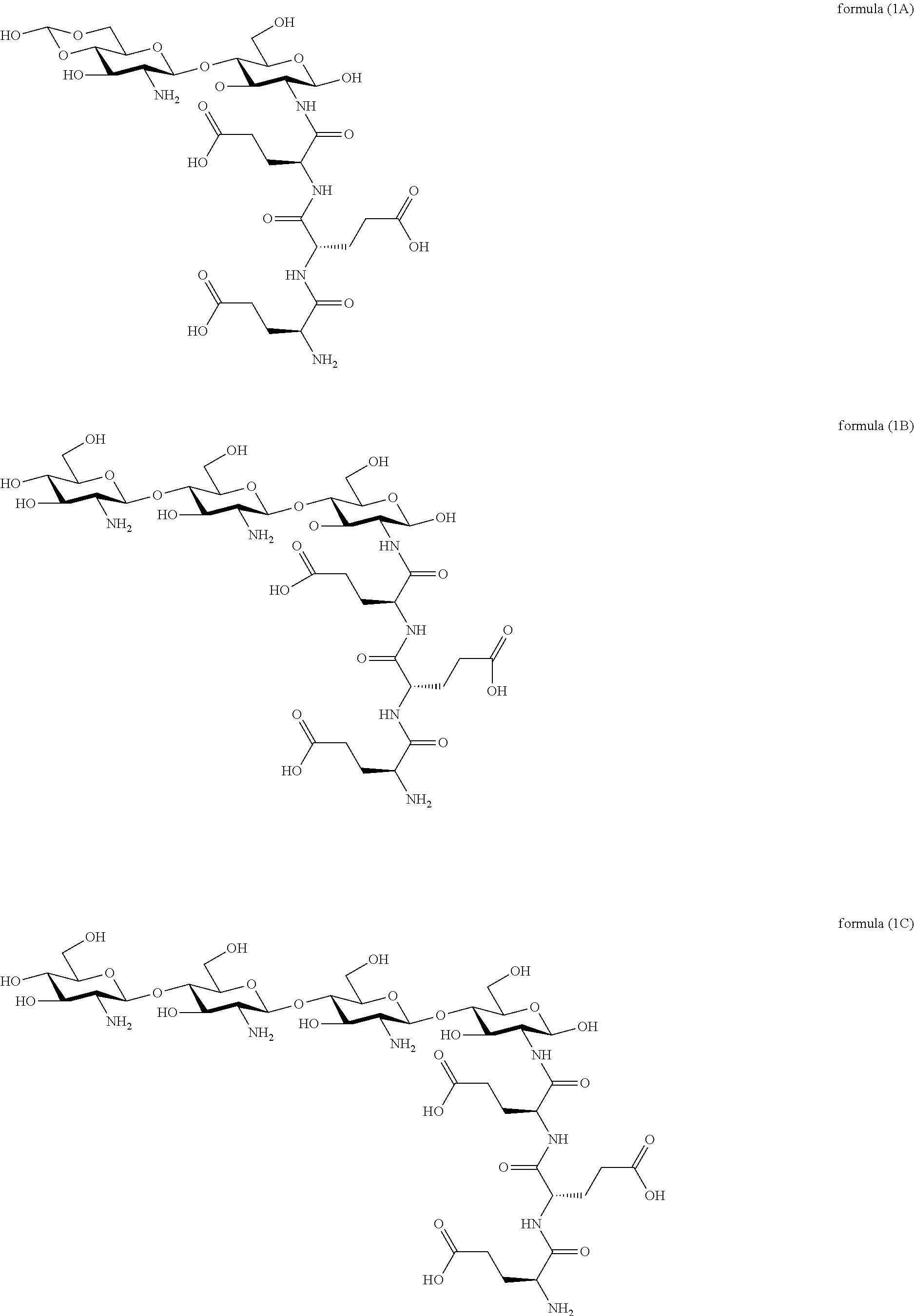

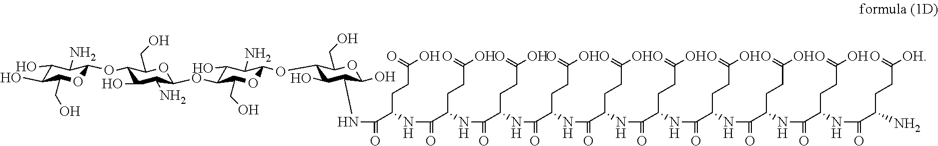

In accordance with another embodiment of the invention, a method of synthesizing a glycopeptide composition is described. The method includes the following steps. A plurality of glycopeptides is formed in the glycopeptide composition, wherein the plurality of glycopeptides is formed by the following steps. A step of synthesizing poly(glucosamine) is performed as follows: a glucosamine acceptor having more than three different protecting groups located on each glucosamine unit is provided, the glucosamine acceptor is deprotected at a C4 position on one glucosamine unit to reveal a hydroxyl group; a glucosamine donor having more than three different protecting groups located on each glucosamine unit is provided, the glucosamine donor is deprotected at a C1 position on one glucosamine unit to reveal another hydroxyl group, and then further reacted to form a leaving group; the glucosamine donor is conjugated with the glucosamine acceptor to obtain an intermediate product, and the intermediate product is deprotected or further reacted to reveal one free amine group so as to obtain the poly(glucosamine). A poly(glutamic acid) compound is provided, wherein glutamic acids located on side chains of the poly(glutamic acid) compound are protected, and a N-terminal of the poly(glutamic acid) compound is protected and a C-terminal of the poly(glutamic acid) compound has a free acid group. The poly(glutamic acid) compound is conjugated to the poly(glucosamine) by coupling the free amine group to the free acid group so as to form the glycopeptide composition having a plurality of glycopeptides.

In an embodiment of the invention, the poly(glucosamine) has 2 to 8 glucosamine units and the poly(glutamic acid) compound has 3 to 20 glutamic acid units after deprotecting all of the remaining protecting groups.

In an embodiment of the invention, the poly(glucosamine) has four glucosamine units and the poly(glutamic acid) compound has ten glutamic acid units after deprotecting all of the remaining protecting groups.

In an embodiment of the invention, the different protecting groups located on the glucosamine acceptor are benzyl protecting group, benzoyl protecting group, acetyl protecting group and silyl protecting group, and the acetyl protecting group located at a C4 position of one of the glucosamine unit is deprotected so as to expose the hydroxyl group.

In an embodiment of the invention, the poly(glutamic acid) compound is conjugated to the poly(glucosamine) via an amide bond.

In an embodiment of the invention, after deprotecting the glycopeptide, the glycopeptide formed is one compound selected from the list of compounds represented by formula (1A) to formula (1D):

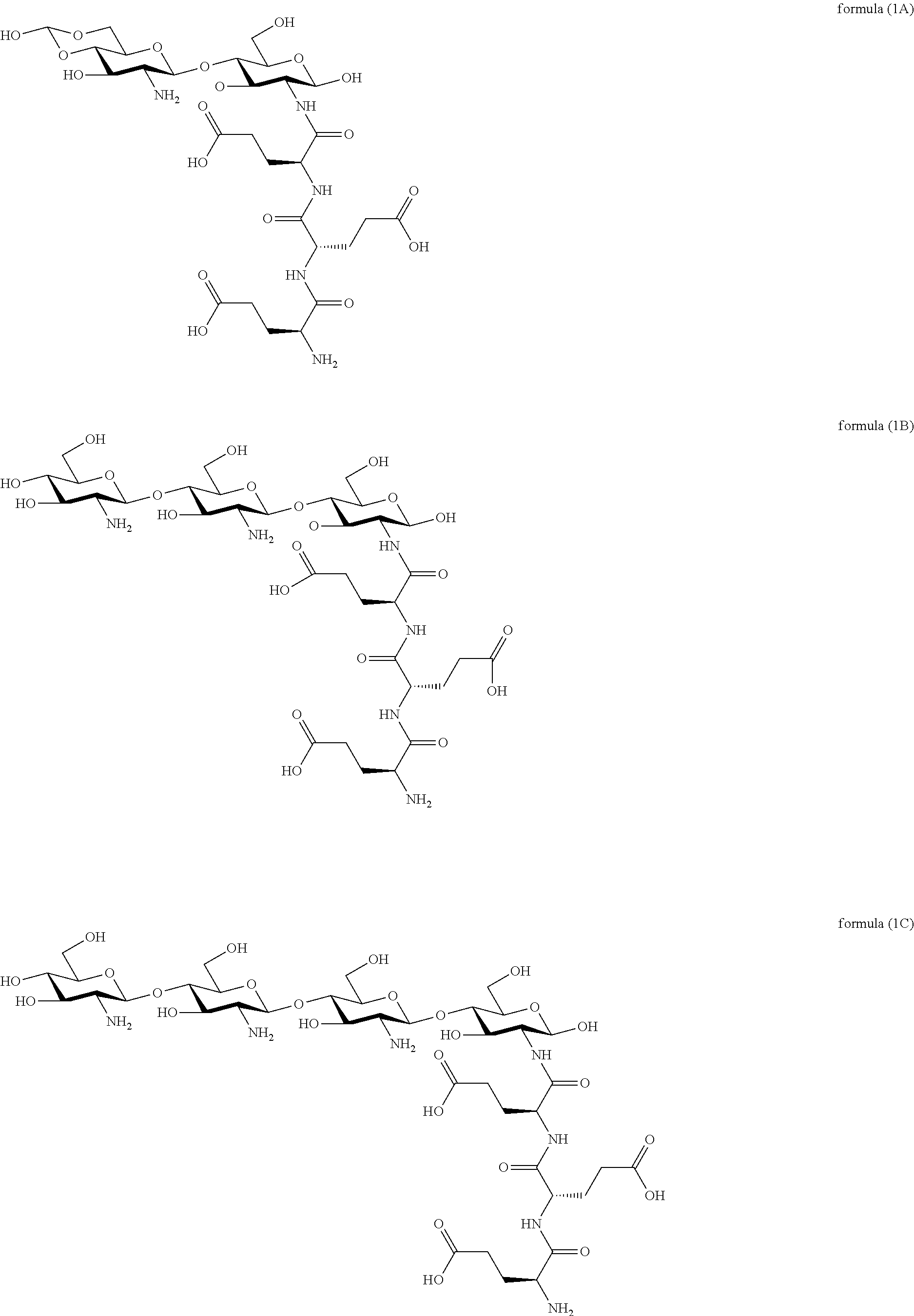

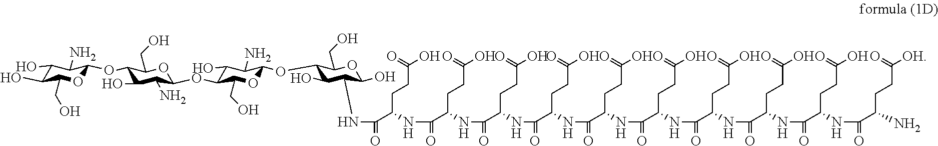

In the glycopeptide composition of the invention, since the polysaccharide moiety is covalently bounded to the peptide moiety via a fixed connection point, therefore, each of the glycopeptide synthesized are connected in the same way, and may have the same number of saccharide units. As such the glycopeptide synthesized will be desirable for drug delivery, or suitable be used as biomaterials, for example, the glycopeptide may be further conjugated with a therapeutic agent or diagnostics and be used for biomedical applications.

To make the aforementioned more comprehensible, several embodiments are described in detail as follows.

### DESCRIPTION OF THE EMBODIMENTS

A glycopeptide composition in one embodiment of the invention comprises a plurality of glycopeptides, and a therapeutic agent or diagnostic conjugated to each of the glycopeptide. Each of the glycopeptides is, for example, obtained by conjugating polysaccharide moieties to peptide moieties. A general scheme for synthesizing glycopeptides is shown in scheme 1 below.

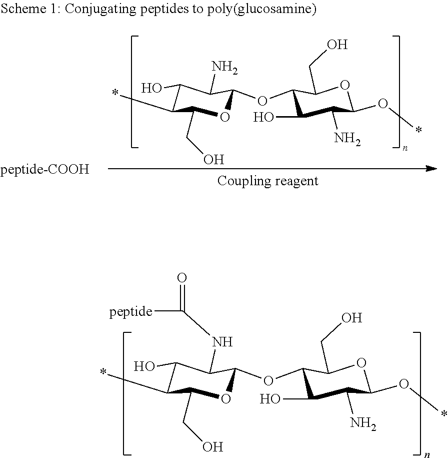

As shown in scheme 1, the peptide moiety is conjugated to poly(glucosamine) (polysaccharide moiety) by the coupling of acid groups (COOH) to amine groups (NH₂) to form an amide bond. However, due to the presence of multiple amine groups in poly(glucosamine), the peptide may be conjugated to poly(glucosamine) at various points, resulting in glycopeptides connected in different ways.

In order to prevent the other amine groups or hydroxyl groups on the poly(glucosamine) (polysaccharide moiety) from reacting, protecting groups (or other suitable leaving groups) are added onto poly(glucosamine) so that only one free amine is available for coupling to peptide. That is, when the poly(glucosamine) has n glucosamine units, then an amount of protected amine groups on the poly(glucosamine) is n−1. Furthermore, the conventional ways for synthesizing poly(glucosamine) usually results in compounds having different chain lengths (monomer units). Therefore, the embodiments of the present disclosure also provide means to control the number of glucosamines units in the poly(glucosamine) polymer.

Specifically, the poly(glucosamine) in an embodiment of the invention can be obtained as follows. A glucosamine acceptor having more than three different protecting groups located on each glucosamine unit is provided, the glucosamine acceptor is deprotected at a C4 position on one glucosamine unit to reveal a hydroxyl group. Subsequently, a glucosamine donor having more than three different protecting groups located on each glucosamine unit is provided, the glucosamine acceptor is deprotected at a C1 position on one glucosamine unit to reveal another hydroxyl group, and then further reacted to form a leaving group. The glucosamine donor is conjugated with the glucosamine acceptor to obtain an intermediate product, and the intermediate product is deprotected or further reacted to reveal one free amine group so as to obtain the poly(glucosamine). Since the poly(glucosamine) is prepared with only one free amine, the connection point of peptides to the poly(glucosamine) can be appropriately controlled.

In one specific embodiment of the invention, a glucosamine monomer with formula (M-1) is provided. The glucosamine monomer with formula (M-1) may be used as the general building block of the poly(glucosamine) polymer.

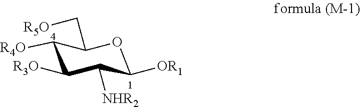

The glucosamine monomer with formula (M-1) may be selectively protected with at least three kinds of different protecting groups or leaving groups. In formula (M-1), R₁ to R₅ may independently be a benzyl (Bn) group, a benzoyl (Bz) group, an acetyl (Ac) group, a silyl (TBS) group, a phthalimide (Phth) group, a tert-butyldimethylsilyl (TBDMS) group, a thio-methyl group, a bromide group, a trichloroacetimidate group or a fluoride group; OR₁ may be replaced by thio-toluene (STol), NHR₂ may be replaced with an azide (N₃), R₄ and R₅ together may be connected to each other to form a ring, which may be substituted or unsubstituted. However, in formula (M-1), the protecting group R₁ at the C1 position is different to the protecting group R₄ at the C4 position, and R₁ to R₅ at least have three kinds of different protecting groups or substituents. Based on the compounds with formula (M-1), the protecting group R₁ and the protecting group R₄ may be removed by different methods.

More specifically, a glucosamine monomer with formula (M-2) is provided.

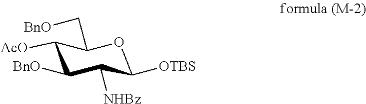

In certain embodiments, the glucosamine monomer with formula (M-2) is protected with at least four different kinds of protecting groups. For example, the glucosamine monomer is protected with a benzyl (Bn) protecting group (in the R₃ and R₅ position), a benzoyl (Bz) protecting group (in the R₂ position), an acetyl (Ac) protecting group (in the R₄ position) and a silyl (TBS) protecting group (in the R₁ position). The different protecting groups allows the poly(glucosamine) to be selectively deprotected to reveal one amine group or one hydroxyl group at a time, which allows for further reaction. By using the glucosamine monomer with formula (M-2), glucosamine donors and glucosamine acceptors may be prepared as shown in Scheme 2 below.

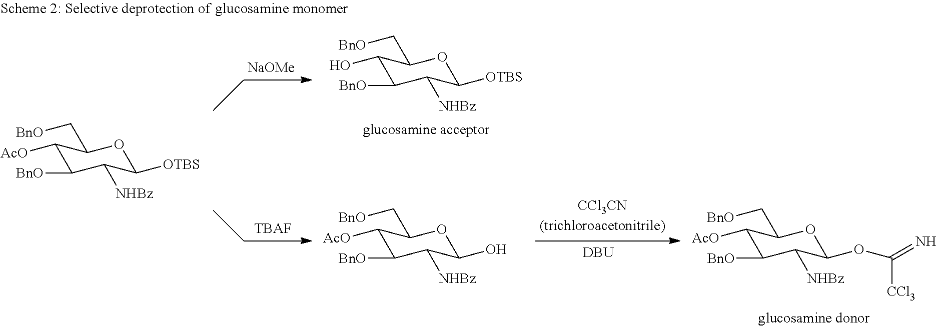

As shown in Scheme 2, the glucosamine monomer may be selectively deprotected by sodium methoxide (NaOMe) to remove the acetyl (Ac) protecting group at the C4 position to obtain a glucosamine having a free hydroxyl group (glucosamine acceptor). Furthermore, the glucosamine monomer may be selectively deprotected by tetrabutylammonium fluoride (TBAF) to remove the silyl (TBS) protecting group at the C1 position to obtain a glucosamine having a free hydroxyl group, then further reacted with trichloroacetonitrile to form a trichloroacetimide leaving group (glucosamine donor). The glucosamine donor and glucosamine acceptor shown in Scheme 2 may be reacted or conjugated to each other to obtain an intermediate product, wherein the intermediate product may be deprotected or further reacted to reveal one free amine group so as to obtain a poly(glucosamine) for reaction/conjugation with peptides. In the exemplary embodiment above, although a poly(glucosamine) having two glucosamine units is used as an example, however, this construes no limitation in the invention. In some other embodiments, the number of glucosamine units in the poly(glucosamine) is in a range from 2 to 8. Furthermore, although poly(glucosamine) was used as a specific example of the polysaccharide moiety, however, this construes no limitation in the invention. In some other embodiments, the polysaccharide moiety may be poly(aspartic acid) or some other types of suitable polysaccharides.

In another embodiment, in order to synthesize poly(glucosamine) having more than two glucosamine units, the method shown in Scheme 3 may be applied.

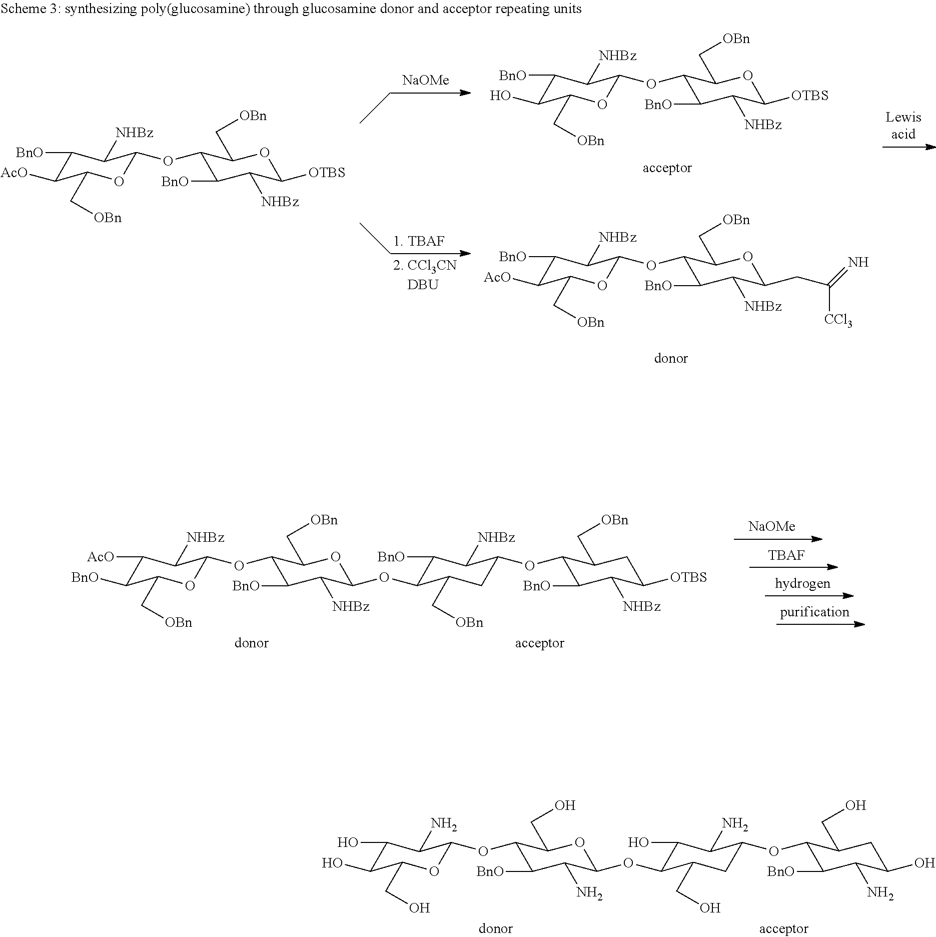

As shown in Scheme 3, the glucosamine donor and the glucosamine acceptor obtained in scheme 2 may be reacted with each other to form a poly(glucosamine) polymer having two glucosamine units. The poly(glucosamine) polymer having two glucosamine units may be further treated as a new monomer (new building block) and be reacted with other monomer units. For example, as shown in Scheme 3, the new monomer (with two glucosamine unit) is selectively deprotected by sodium methoxide (NaOMe) to remove the acetyl (Ac) protecting group at the C4 position of one of the glucosamine unit to obtain a free hydroxyl group (glucosamine acceptor). Furthermore, the new monomer (with two glucosamine unit) may be selectively deprotected by tetrabutylammonium fluoride (TBAF) to remove the silyl (TBS) protecting group at the C1 position of one of the glucosamine unit to obtain a free hydroxyl group, and then further reacted with trichloroacetonitrile to form a trichloroacetimide leaving group (glucosamine donor). The two new monomers, one of which is a glucosamine donor and the other being a glucosamine acceptor, then may be reacted together to form a poly(glucosamine) with four glucosamine units. In some other embodiments, the glucosamine acceptor with two glucosamine unit may be reacted with the glucosamine donor with one glucosamine unit (shown in Scheme 2) to form a poly(glucosamine) with three glucosamine units.

Based on the above mechanism, through the utilization of glucosamine donor and acceptor repeating units, it would be possible to achieve a poly(glucosamine) polymer having the desired number of glucosamine units depending on requirement. Furthermore, as shown in Scheme 3, when the poly(glucosamine) polymer with the desired chain length (glucosamine units) has been achieved, all of the protecting groups located on each of the glucosamine units may be deprotected to reveal the hydroxyl and amine functional groups. However, in situations where the poly(glucosamine) is further used for coupling with a peptide, then the poly(glucosamine) is only deprotected after coupling.

In the embodiments of the present invention, poly(glutamic acid) is generally used as the peptide moiety for coupling with the polysaccharide moiety. However, the invention is not limited thereto, and other types of peptides may be used. In some embodiments, when poly(glutamic acid) is used as the peptide moiety, then a number of glutamic acid units in the poly(glutamic acid) is in a range from 3 to 20. In the case when poly(glucosamine) is coupled with poly(glutamic acid), it is ensured that only one free amine group on the poly(glucosamine) is available for coupling to the poly(glutamic acid), and only one free acid group on the poly(glutamic acid) is available for coupling to the poly(glucosamine). In certain embodiments, the one free acid group on the poly(glutamic acid) is located on a C-terminal of the poly(glutamic acid), such that the poly(glucosamine) is covalently bounded to the C-terminal of the poly(glutamic acid) via an amide bond. As such, the glycopeptide (having the polysaccharide moiety and the peptide moiety) synthesized will be desirable for drug delivery, or suitable be used as biomaterials, for example, the glycopeptide may be further conjugated with a therapeutic agent or diagnostics and be used for biomedical applications.

In one embodiment, the glycopeptide may be conjugated with a metal such as ^{99m}Tc to form a metal complex. An example of the glycopeptide conjugated with a metal is represented by the compound of formula (2). Since the glycopeptide of the invention may be easily taken up/absorbed by cancer cells, and the metal ^{99m}Tc is used for radiolabeling, therefore, the formed metal complex may serve as a diagnostic, and be effectively used as a contrast agent for tumor imaging.

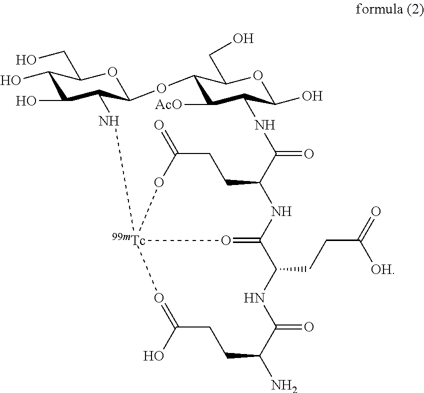

### EXAMPLES

### Example 1

A method for synthesizing a glycopeptide represented by formula (1A) having two glucosamine units and three glutamic acid units is described in the present example.

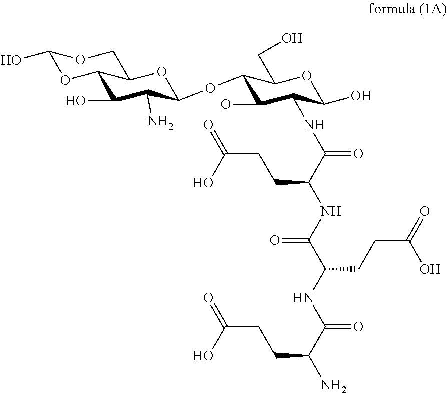

The glycopeptide of formula (1A) can be synthesized by Scheme 4 shown below:

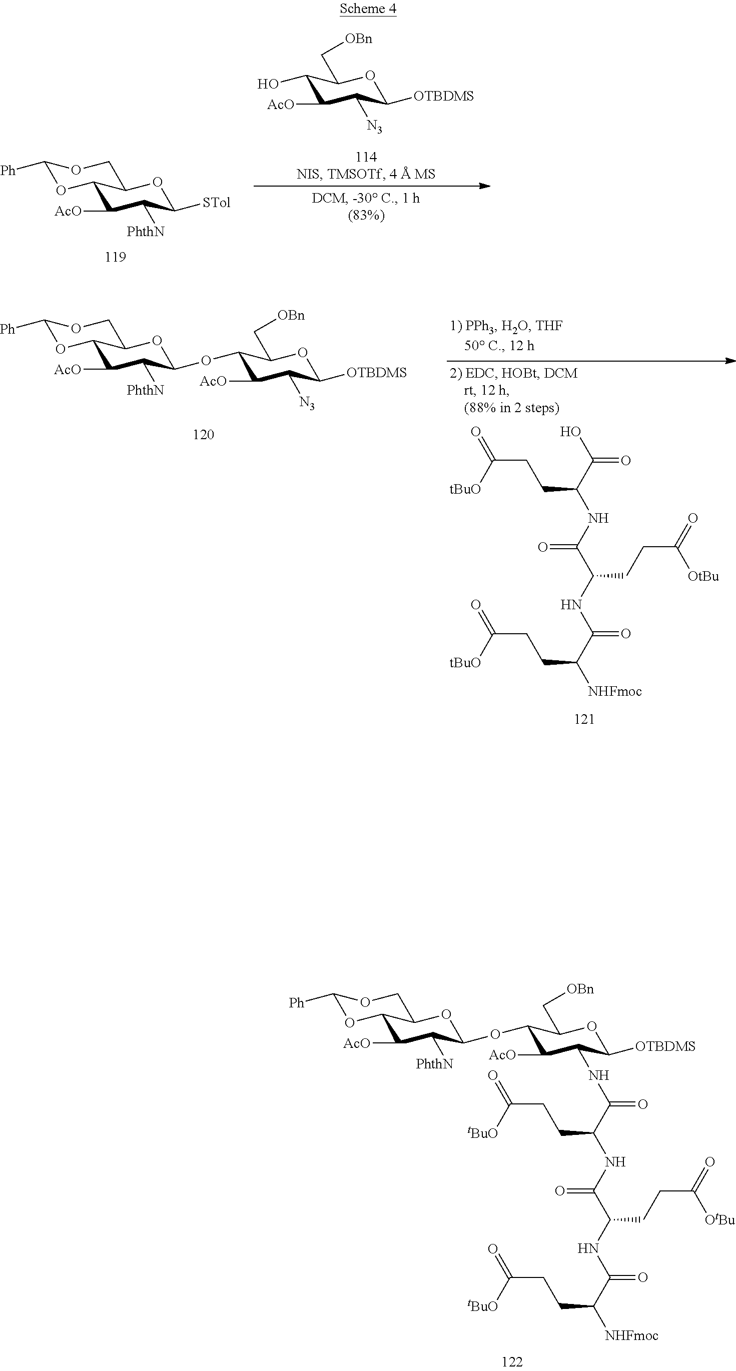

As shown in scheme 4, to a solution of glucosamine acceptor (compound 114) (105 mg, 0.233 mmol) and glucosamine donor (compound 119) (152 mg, 0.279 mmol) in dichloromethane (1 mL), 4 Å molecular sieve (200 mg) was added under nitrogen atmosphere. The resulting solution was stirred for 30 min and cooled to −30° C. and N-iodosuccinimide (NIS) (73 mg, 0.326 mmol) was added under nitrogen atmosphere followed by addition of trimethylsilyl trifluoromethanesulfonate (TMSOTf) (4 μL, 0.023 mmol). The reaction was stirred for 1 hour at −30° C., and the reaction was monitored by thin-layer chromatography (TLC). After completion of the reaction, the resulting mixture was neutralized by triethylamine and reaction mixture was filtered through cellite pad. The solvent was removed under vacuum. The residue was purified by flash column chromatography on silica gel to afford the product (compound 120) (168 mg, 83%).

In a next step, to a solution of compound 120 (81 mg, 0.093 mmol) in tetrahydrofuran (1.6 mL), triphenylphosphine (49 mg, 0.186 mmol) was added at 0° C. and then the reaction mixture was stirred at 0° C. for 5 min. After addition of water (8 μL, 0.419 mmol) at 0° C., the reaction mixture was heated at 50° C. for 12 hours and then concentrated under reduced pressure to obtain a solution having only one free amine on each poly(glucosamine) that is available for reaction (azide converted to amine). Without further purification, the solution of poly(glucosamine) was added into dichloromethane (1.6 mL), and peptide (compound 121) (85 mg, 0.112 mmol), 1-hydroxybenzotriazole (31 mg, 0.233 mmol) and 1-ethyl-3-(3-dimethylaminopropyl)carbodiimide (36 mg, 0.233 mmol) were added successively. The reaction mixture was stirred for 12 hours at 25° C. and then concentrated under reduce pressure, then purified by column chromatography on silica gel to give compound 122 (133 mg, 88% in 2 steps). By using the methods above, a glycopeptide having two glucosamine units and three glutamic acid units can be prepared. The glycopeptide may be fully deprotected to reveal all the functional groups and be conjugated to a therapeutic agent or diagnostic as required.

### Example 2

A method for synthesizing a glycopeptide represented by formula (1B) having three glucosamine units and three glutamic acid units is described in the present example.

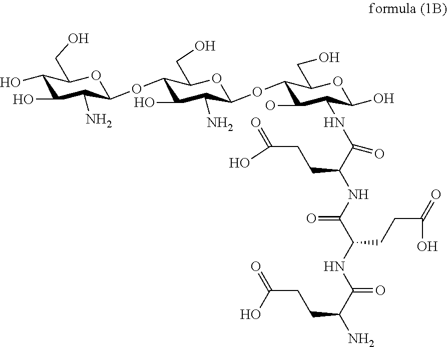

The glycopeptide of formula (1B) can be synthesized by Scheme 5 shown below:

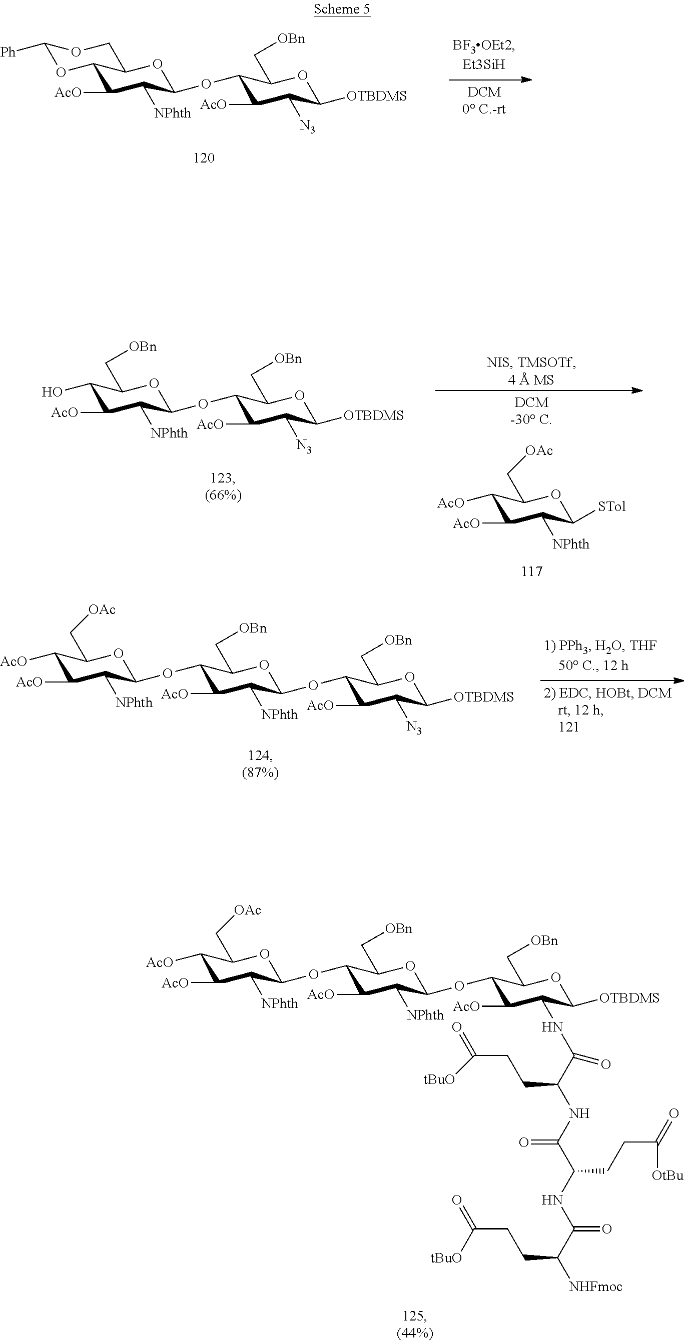

As shown in scheme 5, the compound 120 prepared in Example 1 was used as the starting compound. To the solution of starting compound 120 (870 mg, 0.997 mmol) in anhydrous DCM (20.0 mL), Et₃SiH (250 μL, 10.97 mmol) was added followed by BF₃.OEt₂ (250 μL, 1.99 mmol) was added at 0° C. under N₂. The reaction was allowed to warm, and further stirred at 20° C. for 3 hours. After completion, reaction was quenched by aq. NaHCO₃ solution and water work up was carried out. Crude product was purified by column chromatography using 0-50% EtOAc/hexane to afford (575 mg, 66%) of desired product of compound 123 as white solid.

In a next step, to a suspension of the glucosamine acceptor (compound 123) (1.4 g, 1.60 mmol), glucosamine donor (compound 117) (1.21 g, 2.24 mmol) and molecular sieves 4 Å (1.5 g) in DCM (40 mL) was stirred at room temperature under a N₂ atmosphere for 60 min. Reaction mixture was cooled to −30° C. and NIS (576 mg, 2.56 mmol) was added slowly followed by TMSOTf (71 μL, 0.32 mmol). Reaction mixture was stirred for 2 h at the same temperature and slowly warmed to room temperature. After completion, reaction mixture was filtered through celite pad, filtrate was treated with Na₂S₂O₃ and NaHCO₃. Organic layer was dried over MgSO₄ and concentrated. The crude was purified by column chromatography using silica gel and 0-30% EtOAc/hexane to afford product (1.8 g, 87%) of compound 124 as white solid.

To a solution of compound 124 (65 mg, 0.050 mmol) in THF (3 mL), PPh₃ (26 mg, 0.100 mmol) was added followed by addition of water (4 μL, 0.251 mmol) at 0° C. under a N₂ atmosphere. The reaction mixture was stirred for 2 h at the same temperature and slowly warmed to room temperature. After completion, reaction solvent was removed under reduced pressure. To the solution of above crude in DCM (3 mL), EDC.HCl (19 mg, 0.125 mmol), HOBt (17 mg, 0.125 mmol), TEA (17 μL, 0.125 mmol), were added successively followed by addition of peptide (compound 121) (45 mg, 0.060 mmol). Reaction mixture was stirred for 12 h at the same temperature. After completion, aq. NaHCO₃ was added to reaction mixture and product extracted with DCM (10×3 mL). Organic layer was dried over MgSO₄ and concentrated. The crude was purified by column chromatography using silica gel and 0-30% EtOAc/hexane to afford the desired product of compound 125 (45 mg, 44%) as white solid. By using the methods above, a glycopeptide having three glucosamine units and three glutamic acid units can be prepared. The glycopeptide may be fully deprotected to reveal all the functional groups and be conjugated to a therapeutic agent or diagnostic as required.

### Example 3

A method for synthesizing a glycopeptide represented by formula (1C) having four glucosamine units and three glutamic acid units is described in the present example.

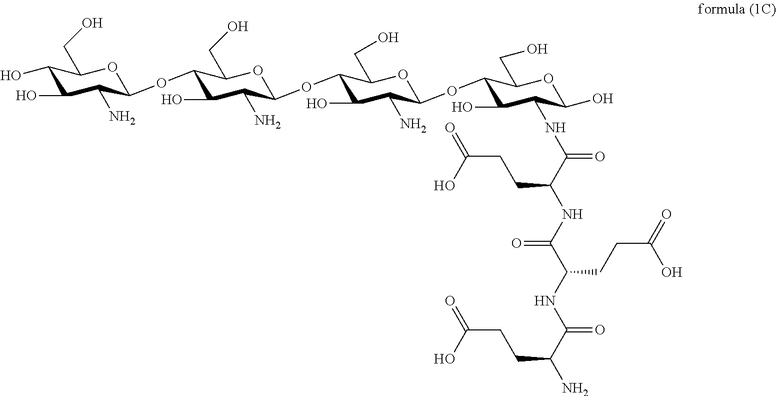

The glycopeptide of formula (1C) can be synthesized by Scheme 6 shown below:

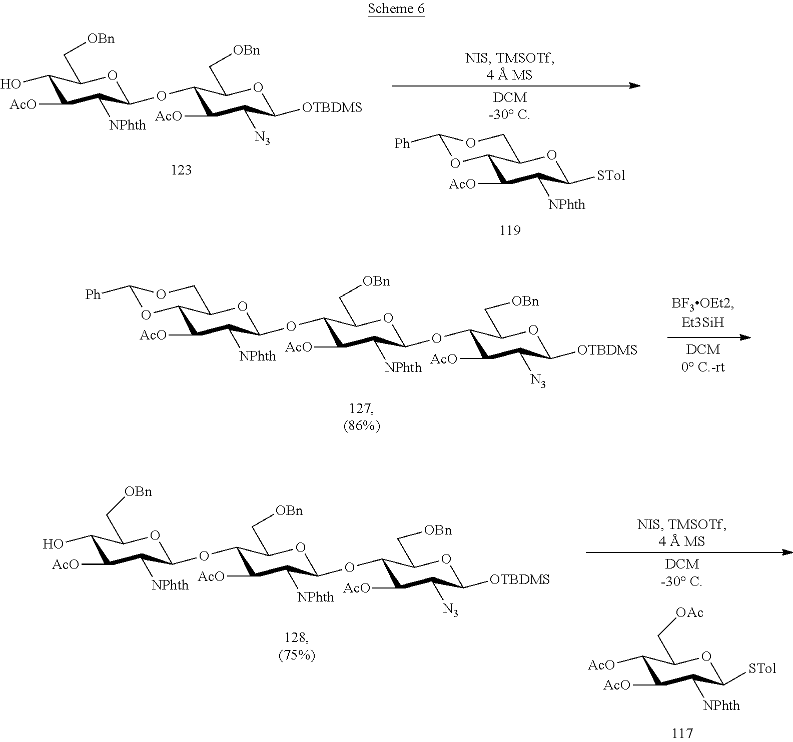

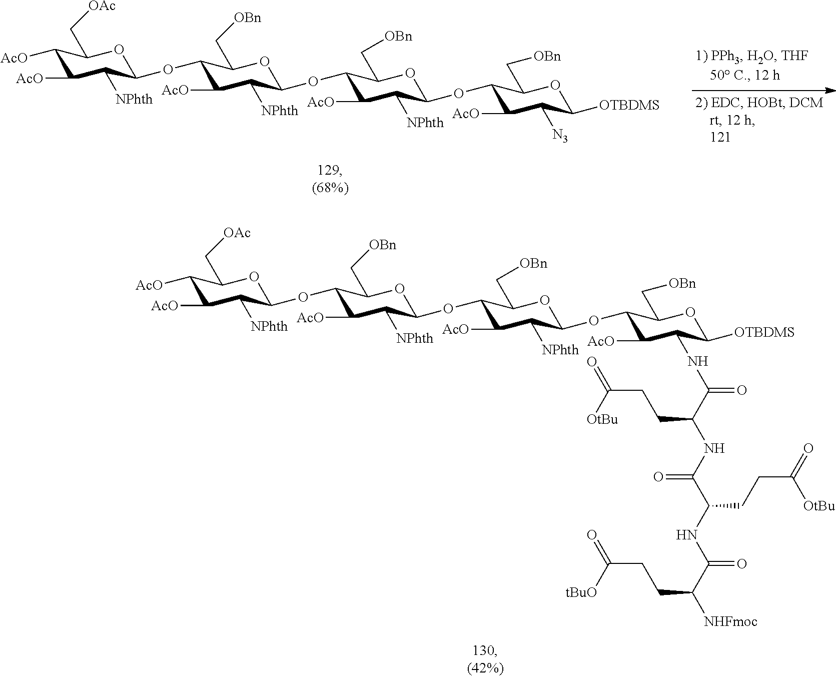

As shown in scheme 6, the compound 123 prepared in Example 2 was used as the starting compound. To the solution of glucosamine acceptor (compound 123) (40 mg, 0.045 mmol) and glucosamine donor 119 (29 mg, 0.054 mmol) in dichloromethane (2.5 mL), 4 Å molecular sieve (100 mg) was added under nitrogen atmosphere. The resulting solution was stirred for 60 min and cooled to −30° C. N-iodosuccinimide (14 mg, 0.064 mmol) was added under nitrogen atmosphere followed by addition of trimethylsilyl trifluoromethanesulfonate (1.0 μL, 0.004 mmol). The reaction was stirred for 2 hour at −30° C., and monitored by thin-layer chromatography. After completion, reaction mixture was filtered through celite pad, filtrate was treated with Na₂S₂O₃ and NaHCO₃. Organic layer was dried over MgSO₄ and concentrated. The crude was purified by column chromatography using silica gel and 0-40% EtOAc/hexane to afford (51 mg, 86%) of desired product of compound 127 as white solid. To the solution of compound 127 in anhydrous DCM (20.0 mL) Et₃SiH (250 μL, 10.97 mmol) was added followed by BF₃.OEt₂ (250 μL, 1.99 mmol) was added at 0° C. under N₂. The reaction was allowed to warm and further stirred at 20° C. for 3 hours. After completion, reaction was quenched by aq. NaHCO₃ solution and water work up was carried out. Crude product was purified by column chromatography using 0-50% EtOAc/hexane to afford the desired product of compound 128 as white solid.

In a next step, to a suspension of the glucosamine acceptor (compound 128) (280 mg, 0.215 mmol), glucosamine donor (compound 117) (163 mg, 0.302 mmol) and molecular sieves 4 Å (0.5 g) in DCM (7.0 mL) was stirred at room temperature at N₂ atmosphere for 60 minutes. The reaction mixture was cooled to −30° C. and NIS (77 mg, 0.345 mmol) was added slowly followed by TMSOTf (11 μL, 0.043 mmol). Reaction mixture was stirred for 2 h at the same temperature and slowly warmed to room temperature. After completion, reaction mixture was filtered through celite pad, filtrate was treated with Na₂S₂O₃ and NaHCO₃. Organic layer was dried over MgSO₄ and concentrated. The crude was purified by column chromatography using silica gel and 0-40% EtOAc/hexane to afford the product of compound 129 (251 mg, 68%) as white solid.

Subsequently, to a solution of compound 129 (230 mg, 0.134 mmol) in THF, PPh₃ (70 mg, 0.268 mmol) was added at 0° C. followed by H₂O (12 μL, 0.67 mmol) and the resulting solution was stirred at room temperature under a N₂ atmosphere for overnight. After completion, reaction solvent was removed under vacuum and crude (poly(glucosamine) having one free amine) was kept under high vacuum. To the above crude in DCM (5 mL), peptide (compound 121) (122 mg, 0.161 mmol), EDC (52 mg, 0.335 mmol), HOBt (45 mg, 0.335 mmol), TEA (45 μL, 0.335 mmol) were added successively at 0° C. Reaction was allowed to warm to rt. After completion, reaction was quenched by water. Product was extracted with DCM (20 mL×3). The crude was purified by column chromatography using silica gel and 0-80% EtOAc/hexane as gradient to afford the desired product of compound 130 (140 mg, 42%) as white solid. By using the methods above, a glycopeptide having three glucosamine units and three glutamic acid units can be prepared. The glycopeptide may be fully deprotected to reveal all the functional groups and be conjugated to a therapeutic agent or diagnostic as required.

### Example 4

A method for synthesizing a glycopeptide represented by formula (1D) having four glucosamine units and ten glutamic acid units is described in the present example.

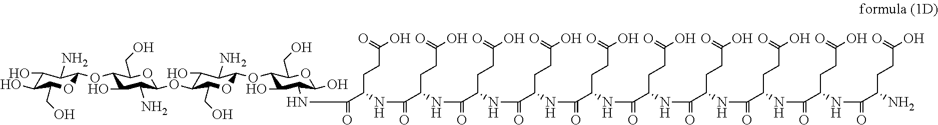

The glycopeptide of formula (1C) can be synthesized by Scheme 7 shown below:

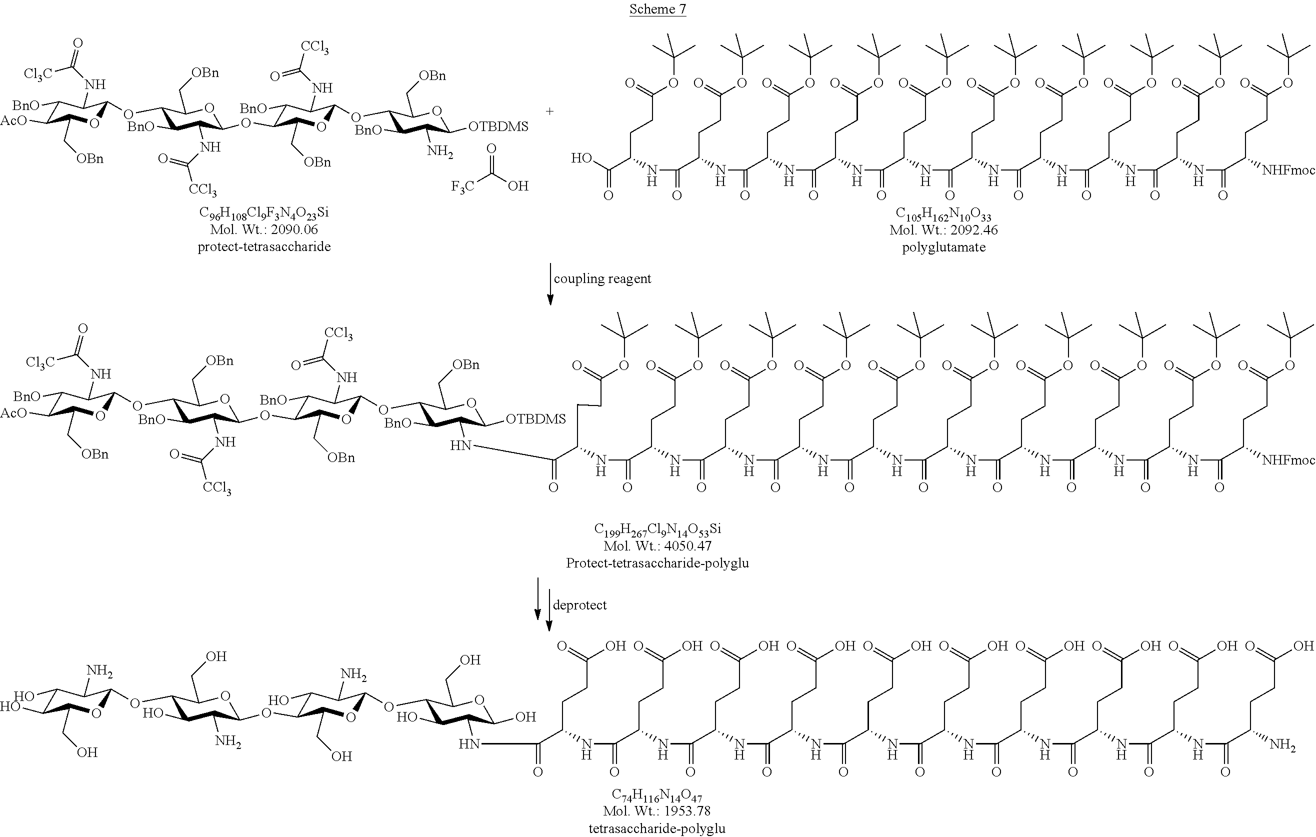

As shown in Scheme 7, a poly(glucosamine) polymer with four glucosamine units (protected tetrasaccharide) is provided, and a poly(glutamic acid) compound with ten protected glutamic acid groups (glutamate) is provided. The poly(glucosamine) polymer may for example, be prepared by referring to the synthesis of compound 129 in Example 3, or by referring to Scheme 3 for a more generalized method of synthesizing poly(glucosamine) having four glucosamine units. By using a specific coupling agent (such as EDC.HCl, HOBt, Triethyl amine/CH₂Cl₂), the poly(glucosamine) polymer with four glucosamine units and the poly(glutamic acid) compound with ten protected glutamic acid groups (glutamate) maybe coupled together to form a protected glycopeptide (protected tetrasaccharide-polyglutamate). More specifically, poly(glucosamine) is covalently bounded to the C-terminal of the poly(glutamic acid) via an amide bond. After coupling, all of the remaining protecting groups located on the poly(glucosamine) polymer and the poly(glutamic acid) compound maybe deprotected to give a glycopeptide of the invention. The deprotection may be achieved by four steps, for example, by (1) using AcOH/Zn for the deprotection of Cl₃CCO group, (2) using Pd/C for the deprotection of OBn group, (3) using Piperidine for the deprotection of the Fmoc group, and (4) using TFA, Et₃SiH for the deprotection of the the t-butyl groups on the peptides.

In the above embodiments, the poly(glucosamine) polymer with multiple glucosamine units, only have one free amine group on the poly(glucosamine) that is available for coupling to the poly(glutamic acid), while the remaining functional groups are protected. As for the poly(glutamic acid) compound, only one free acid group located on a C-terminal of the poly(glutamic acid) is available for coupling to the poly(glucosamine), while the remaining functional groups are protected. Therefore, in the glycopeptide composition of the invention, when conjugating the polysaccharide moiety to the peptide moiety, each of the glycopeptide synthesized are connected in the same way (via a fixed connection point), and may have the same number of saccharide units. As such, the chain lengths and structure of the glycopeptide may be appropriately tuned, and a glycopeptide composition that is desirable for drug delivery, or suitable to be used as biomaterials can be achieved. For example, the glycopeptide may be further conjugated with a therapeutic agent or diagnostics and be used for biomedical applications.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A glycopeptide composition, the glycopeptide composition comprises:
a plurality of glycopeptides, wherein each of the glycopeptide comprises a polysaccharide moiety connected to a peptide moiety, and the polysaccharide moiety is covalently bounded to the peptide moiety via a fixed connection point, wherein the fixed connection point is the same in each of the glycopeptide; and
a therapeutic agent or diagnostic conjugated to each of the glycopeptide.

2. The glycopeptide composition according to claim 1, wherein the peptide moiety is poly(glutamic acid) or poly(aspartic acid).

3. The glycopeptide composition according to claim 2, wherein the polysaccharide moiety is poly(glucosamine) and the peptide moiety is poly(glutamic acid).

4. The glycopeptide composition according to claim 3, wherein the poly(glucosamine) is connected to the poly(glutamic acid) via an amide bond.

5. The glycopeptide composition according to claim 3, wherein a number of glucosamine units in the poly(glucosamine) is in a range from 2 to 8.

6. The glycopeptide composition according to claim 3, wherein a number of glutamic acid units in the poly(glutamic acid) is in a range from 3 to 20.

7. The glycopeptide composition according to claim 3, wherein the poly(glucosamine) has four glucosamine units and the poly(glutamic acid) has ten glutamic acid units.

8. The glycopeptide composition according to claim 3, wherein when the poly(glucosamine) has n glucosamine units, then an amount of protected amine groups on the poly(glucosamine) is n−1.

9. The glycopeptide composition according to claim 3, wherein only one free amine group on the poly(glucosamine) is available for coupling to the poly(glutamic acid), and only one free acid group on the poly(glutamic acid) is available for coupling to the poly(glucosamine).

10. The glycopeptide composition according to claim 9, wherein the one free acid group on the poly(glutamic acid) is located on a C-terminal of the poly(glutamic acid), such that the poly(glucosamine) is covalently bounded to the C-terminal of the poly(glutamic acid) via an amide bond.

11. The glycopeptide composition according to claim 1, wherein after deprotecting the glycopeptide, the glycopeptide is one compound selected from the list of compounds represented by formula (1A) to formula (1D):

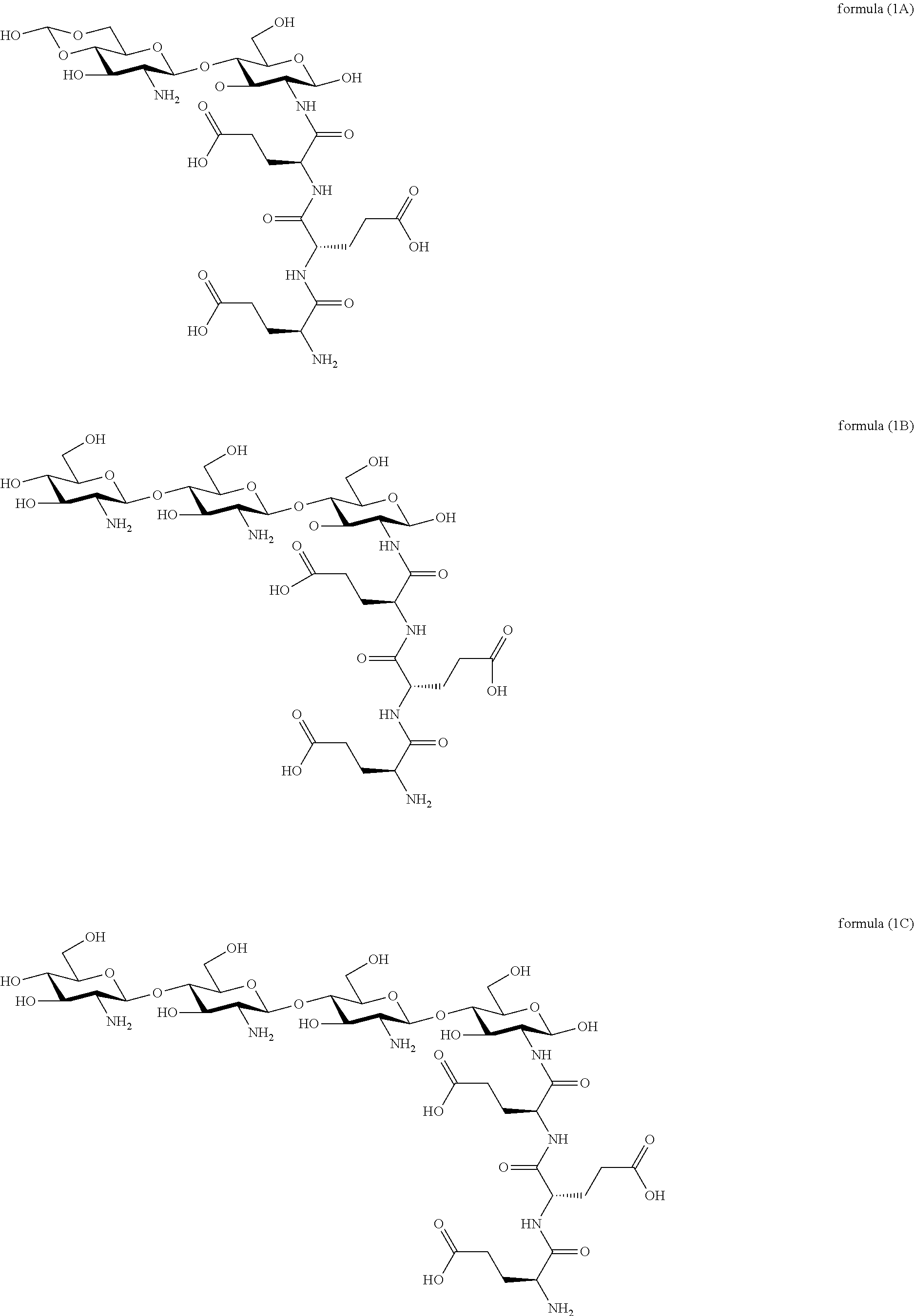

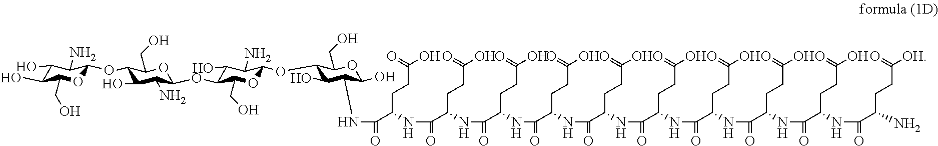

12. A method of synthesizing a glycopeptide composition, comprising:
forming a plurality of glycopeptides in the glycopeptide composition, wherein the forming of the plurality of glycopeptides comprises:
a step of synthesizing poly(glucosamine), wherein the step of synthesizing the poly(glucosamine) comprises:
providing a glucosamine acceptor having more than three different protecting groups located on each glucosamine unit, and deprotecting the glucosamine acceptor at a C4 position on one glucosamine unit to reveal a hydroxyl group;
providing a glucosamine donor having more than three different protecting groups located on each glucosamine unit, deprotecting the glucosamine donor at a C1 position on one glucosamine unit to reveal another hydroxyl group, and then further reacting the another hydroxyl group to form a leaving group;
conjugating the glucosamine donor to the glucosamine acceptor to obtain an intermediate product, and further deprotecting or reacting the intermediate product to reveal one free amine group so as to obtain the poly(glucosamine);
providing a poly(glutamic acid) compound, wherein glutamic acids located on side chains of the poly(glutamic acid) compound are protected, and a N-terminal of the poly(glutamic acid) compound is protected and a C-terminal of the poly(glutamic acid) compound has a free acid group;
conjugating the poly(glutamic acid) compound to the poly(glucosamine) by coupling the free amine group to the free acid group so as to form the plurality of glycopeptides.

13. The method of synthesizing the glycopeptide composition according to claim 12, wherein the poly(glucosamine) has 2 to 8 glucosamine units and the poly(glutamic acid) compound has 3 to 20 glutamic acid units after deprotecting all of the remaining protecting groups.

14. The method of synthesizing the glycopeptide composition according to claim 12, wherein the poly(glucosamine) has four glucosamine units and the poly(glutamic acid) compound has ten glutamic acid units after deprotecting all of the remaining protecting groups.

15. The method of synthesizing the glycopeptide composition according to claim 12, wherein the different protecting groups located on the glucosamine acceptor are benzyl protecting group, benzoyl protecting group, acetyl protecting group and silyl protecting group, and the acetyl protecting group located at a C4 position of one of the glucosamine unit is deprotected so as to expose the hydroxyl group.

16. The method of synthesizing the glycopeptide composition according to claim 12, wherein the different protecting groups located on the glucosamine donor are benzyl protecting group, benzoyl protecting group, acetyl protecting group and silyl protecting group, and the silyl protecting group located at a C1 position of one of the glucosamine unit is deprotected so as to expose the another hydroxyl group.

17. The method of synthesizing the glycopeptide composition according to claim 12, wherein the poly(glutamic acid) compound is conjugated to the poly(glucosamine) via an amide bond.

18. The method of synthesizing the glycopeptide composition according to claim 12, wherein after deprotecting the glycopeptide, the glycopeptide formed is one compound selected from the list of compounds represented by formula (1A) to formula (1D): 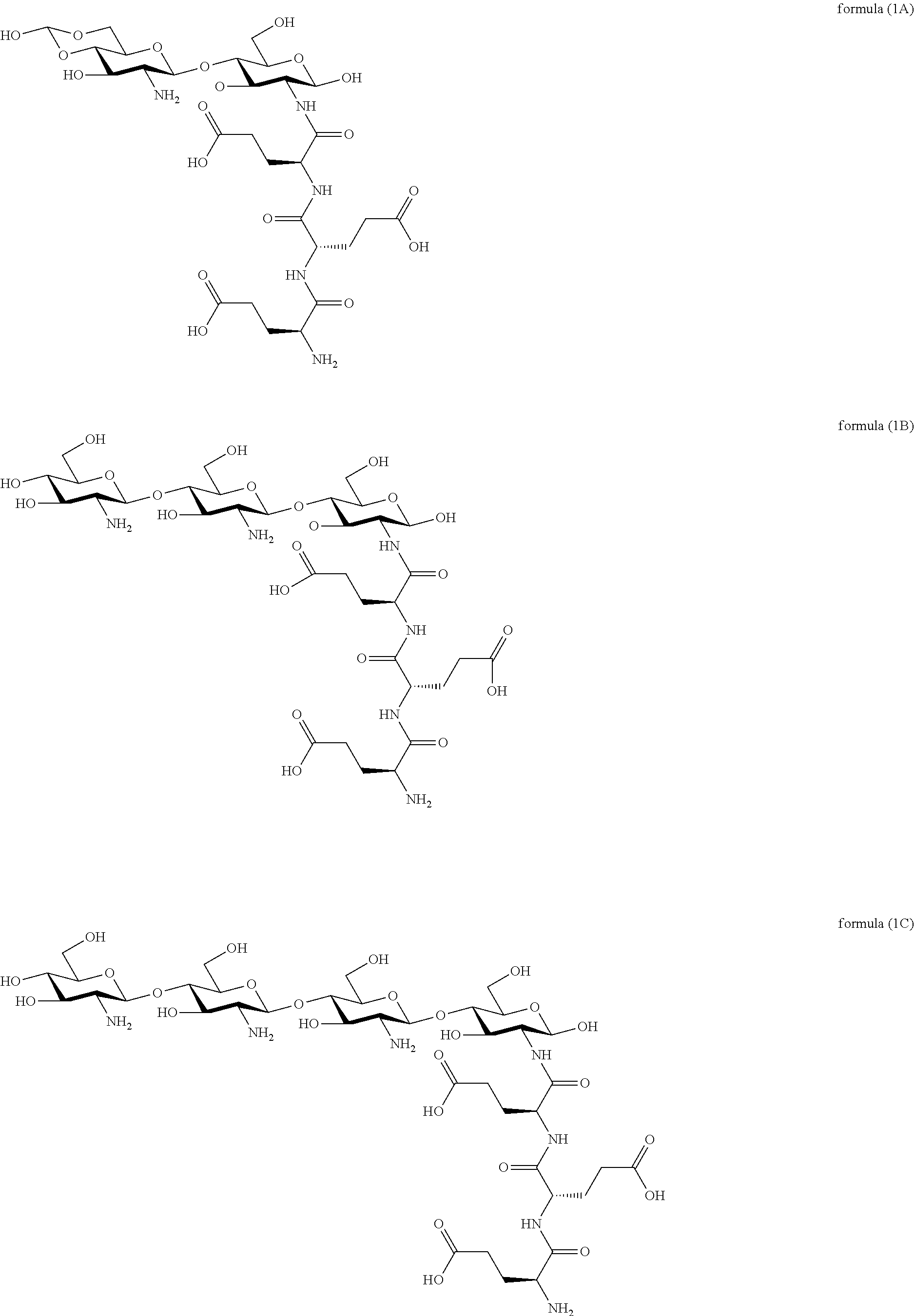 [chemical structure]
